(19) United States
(12) Patent Application Publication
(10) Pub. No.: US 2024/0307281 A1
(43) Pub. Date: Sep. 19, 2024

(54) STABLE FATTY ACID SOAP-TYPE COSMETIC COMPOSITION

(71) Applicant: L V M H RECHERCHE, Saint Jean De Braye (FR)

(72) Inventors: Bora KIM, Chiyoda-ku, Tokyo (JP); Mai OZAWA, Chiyoda-ku, Tokyo (JP)

(73) Assignee: L V M H RECHERCHE, Saint Jean De Braye (FR)

(21) Appl. No.: 18/268,444

(22) PCT Filed: Dec. 22, 2020
(86) PCT No.: PCT/IB2020/001129

§ 371 Date: Jun. 20, 2023

(51) Int. Cl. A61K 8/37; A61K 8/04; A61K 8/44; A61K 8/46; A61K 8/73; A61K 8/81

(52) U.S. Cl. CPC A61K 8/37; A61K 8/046; A61K 8/442; A61K 8/466; A61K 8/731; A61K 8/8129; A61K 8/817; A61K 2800/48

(57) ABSTRACT

A cosmetic composition comprising (a) one or more higher fatty acids of 12 to 24 carbon atoms or their alkali metal salts, (b) at least one cationic polymer selected from the group consisting of polyquaternium-7 and polyquaternium-22, (c) an anionic surfactant and/or an amphoteric surfactant, and (d) at least one thickener selected from the group consisting of hydrophobized hydroxypropyl methylcellulose, and (meth)acrylic-based polymers comprising a (meth)acryloylalkyltaurine or its salt as a structural unit.

### TECHNICAL FIELD

The present invention relates to a cosmetic composition.

### BACKGROUND ART

Fatty acid cream soaps are cosmetics used to remove dirt, dust and excess sebum from the skin by foam formed during their use. Fatty acid cream soaps therefore usually contain surfactants in order to provide satisfactory foaming ability. Mild and foaming skin cleansers compositions are known from Japanese patent applications JP-2014-125434 and JP-2014-125435: these compositions comprise (A) N-acylamino acid, (B) a hydrocarbon oil which is solid or semi-solid at 30° C. including paraffin, petrolatum and the like and (C) a hydroxypropyl starch phosphate with specific mass ratio B/C. In these compositions, the solid or semi-solid hydrocarbon oil at 30° C. can improve the moist feeling, smoothness and stability over time after towel drying, but there is still a need to provide new fatty acid soap-type cosmetic composition without drying of the skin even after use, while maintaining high foaming ability, even without addition of solid or semi-solid hydrocarbon oil at 30° C. including paraffin.

### Technical Problem

Furthermore, because surfactants also have the property of breaking the skin barrier which prevents evaporation of moisture from the skin, using fatty acid cream soaps containing surfactants leads to the problem of greater skin dryness after use. One way of preventing skin dryness after use of a fatty acid cream soap is to add a humectant such as a polyol to the fatty acid cream soap. However, because humectants can interfere with foaming ability it has been difficult to include humectants while also maintaining high foaming ability. Such fatty acid cream soaps have also had stability problems, whereby they tend to separate at high temperature.

It is an object of the invention to provide a stable fatty acid soap-type cosmetic composition without drying of the skin even after use, while maintaining high foaming ability. The inventors developed such a composition by combining specific higher fatty acids, cationic polymers, surfactants and thickeners.

### SUMMARY OF THE INVENTION

So the present invention provides a cosmetic composition comprising (a) one or more higher fatty acids of 12 to 24 carbon atoms or their alkali metal salts, (b) at least one cationic polymer selected from the group consisting of polyquaternium-7 and polyquaternium-22, (c) an anionic surfactant and/or an amphoteric surfactant, and (d) at least one thickener selected from the group consisting of hydrophobized hydroxypropyl methylcellulose, and (meth)acrylic polymers comprising a (meth)acryloylalkyltaurine or its salt as a structural unit. The term “(a) one or more higher fatty acids of 12 to 24 carbon atoms or their alkali metal salts” will also be referred to simply as “component (a)”, and the other components will also be abbreviated similarly. Also, as used herein, the term “(meth)acrylic” refers to acrylic or methacrylic, with the same applying to analogous backbones.

In the cosmetic composition of the invention, the one or more higher fatty acids of 12 to 24 carbon atoms or their alkali metal salts may also be two or more selected from the group consisting of lauric acid, myristic acid, palmitic acid, stearic acid and their alkali metal salts. This will allow even higher foaming ability to be obtained.

The anionic surfactant and/or an amphoteric surfactant in the cosmetic composition of the invention may be one or more selected from the group consisting of alkyl ether carboxylic acid salts, N-acylglutamic acid salts, N-methyl-N-acyltaurine salts, and betaine aminoacetate-type amphoteric surfactants. This will help to inhibit drying of the skin after use.

The hydrophobized hydroxypropyl methylcellulose in the cosmetic composition of the invention may be hydroxypropyl methylcellulose stearoxy ether, and the (meth)acrylic-based polymer may be (hydroxyethyl acrylate/sodium acryloyldimethyltaurate) copolymer. The stability of the cosmetic composition will thus be even more excellent.

The cosmetic composition of the invention may further comprise a nonionic surfactant (e). By comprising component (e), the stability of the cosmetic composition will be even more excellent.

The nonionic surfactant in the cosmetic composition of the invention may be at least one selected from the group consisting of glycerin fatty acid esters and fatty acid polyethylene glycols. This will result in even more excellent stability of the cosmetic composition.

The present invention also relates to a cosmetic process for cleansing keratinic materials, comprising the application onto keratinic materials, in particular onto skin, of the cosmetic composition of the invention. In particular, the composition is further rinsed-off from the keratinic materials.

### DETAILED DESCRIPTION OF THE INVENTION

Preferred embodiments of the invention will now be described, with the understanding that these embodiments are in no way limitative on the invention.

### Higher Fatty Acids

The cosmetic composition of the invention contains (a) one or more higher fatty acids of 12 to 24 carbon atoms or their alkali metal salts.

Examples of higher fatty acids of 12 to 24 carbon atoms include lauric acid, myristic acid, palmitic acid, stearic acid, isostearic acid, arachidic acid, behenic acid, palmitoleic acid, oleic acid, linoleic acid and linolenic acid.

Examples of alkali metal salts of higher fatty acids of 12 to 24 carbon atoms include their sodium salts and potassium salts.

In a particular and preferred embodiment, to allow even higher foaming ability to be obtained, component (a) is preferably a combination of two or more higher fatty acids of 12 to 24 carbon atoms or their alkali metal salts, more preferably a combination of two or more higher fatty acids of 12 to 18 carbon atoms or their alkali metal salts, even more preferably a combination of two types selected from the group consisting of lauric acid, myristic acid, palmitic acid, stearic acid and their alkali metal salts, and most preferably combinations of lauric acid, myristic acid, stearic acid and their alkali metal salts.

In a particular embodiment, the total content of component(s) (a) ranges preferably from 15 to 50 mass % and more preferably from 20 to 40 mass % based on the total mass of the composition. If the content of component (a) is within this range it will be possible to impart even higher foaming ability. As used herein, “based on the total mass” means based on the total mass of the cosmetic composition.

### Cationic Polymer

The cosmetic composition of the invention comprises (b) at least one cationic polymer selected from the group consisting of polyquaternium-7 and polyquaternium-22.

Polyquaternium-7 is a cationic polymer, also known as dimethyldiallylammonium chloride-acrylamide copolymer. Polyquaternium-22 is also a cationic polymer, also known as dimethyldiallylammonium chloride-acrylic acid copolymer.

In a particular and preferred embodiment, Component (b) is polyquaternium-22, to allow drying of the skin after use to be further inhibited.

The total content of component(s) (b) ranges preferably from 0.2 to 3.5 mass % and more preferably from 0.5 to 2.5 mass % based on the total mass of the composition. If the total content of component(s) (b) is within this range it will be possible to even further inhibit drying of the skin after use.

Anionic Surfactant and/or an Amphoteric Surfactant

The cosmetic composition of the invention also comprises (c) an anionic surfactant and/or an amphoteric surfactant. Component (c) may be a single compound used alone, or two or more may be used in combination.

Examples of anionic surfactants according to the invention include carboxylic acid-type anionic surfactants other than component (a), including alkyl ether carboxylic acid salts (such as sodium lauryl glycol carboxylate), polyoxyethylene alkyl ether carboxylic acid salts (such as polyoxyethylene lauryl ether acetate), N-acylsarcosine salts (such as sodium lauroyl sarcosinate and sodium myristoyl sarcosinate), N-acylglutamic acid salts (such as sodium lauroyl glutamate, sodium stearoyl glutamate and sodium cocoyl glutamate); sulfonic acid-type anionic surfactants, including dialkylsulfosuccinic acid salts (such as sodium di-2-ethylhexylsulfosuccinate), alkane sulfonates, α-olefin sulfonates, alkylbenzene sulfonates, alkylnaphthalene sulfonates and N-methyl-N-acyltaurine salts (such as sodium lauroyl methyltaurate, sodium myristoyl methyltaurate and sodium cocoyl methyltaurate); sulfuric acid ester-type anionic surfactants, including alkylsulfuric acid salts (such as sodium lauryl sulfate and sodium myristyl sulfate) and polyoxyethylene alkyl ether sulfuric acid salts (such as sodium polyoxyethylene lauryl sulfate); and phosphate-type anionic surfactants, including alkylphosphoric acid salts (such as sodium lauryl phosphate and potassium cetyl phosphate) and polyoxyethylene alkyl ether phosphates (such as sodium polyoxyethylene stearyl ether phosphate), and mixtures thereof. In a particular and preferred embodiment, alkyl ether carboxylic acid salts such as sodium laurylglycol carboxylate, N-acylglutamic acid salts such as sodium lauroyl glutamate and N-methyl-N-acyltaurine salts such as sodium cocoyl methyltaurate are used to further inhibit drying of the skin after use.

Examples of amphoteric surfactants according to the invention include glycine-type amphoteric surfactants such as sodium cocoamphoacetate and lauroamphoacetic acid; betaine aminoacetate-type amphoteric surfactants such as cocamidopropyl betaine, lauroamidopropyl betaine, cocobetaine and lauryl betaine; sulfobetaine-type amphoteric surfactants such as lauryl hydroxysultaine and lauramidopropyl hydroxysultaine; and lecithins such as lecithin and hydrogenated lecithin, and mixtures thereof. In a particular and preferred embodiment, betaine aminoacetate-type amphoteric surfactants such as cocamidopropyl betaine are used for further inhibiting drying of the skin after use.

The total content of component(s) (c) ranges preferably from 5 to 20 mass % and more preferably from 8 to 15 mass % based on the total mass of the composition. If the total content of component(s) (c) is within this range it will be possible to even further inhibit drying of the skin after use.

### Thickener

The cosmetic composition of the invention also comprises (d) at least one thickener selected from the group consisting of hydrophobized hydroxypropyl methylcellulose, and (meth)acrylic-based polymers comprising a (meth)acryloylalkyltaurine or its salt as a structural unit. Component (d) may be a single one used alone, or two or more may be used in combination.

In a particular embodiment, hydrophobized hydroxypropyl methylcellulose is hydroxypropyl methylcellulose that has had a hydrophobic group (such as alkyl) introduced into the hydroxypropyl group of the hydroxypropyl methylcellulose. An example for the hydrophobized hydroxypropyl methylcellulose according to the invention is hydroxypropyl methylcellulose stearoxy ether having a stearyl group introduced into the hydroxypropyl group of the hydroxypropyl methylcellulose. The hydroxypropyl methylcellulose stearoxy ether used in the present invention may be a commercial product such as SANGELOSE® 60L or SANGELOSE® 90L (both products of Daido Chemical Corporation).

The term “(meth)acrylic-based polymer”, as used herein, means a polymer including a (meth)acryloylalkyltaurine or its salt as a structural unit. Specific examples of (meth)acrylic-based polymers according to the invention include (ammonium acryloyldimethyltaurate/beheneth-25 methacrylate) crosspolymer, (hydroxyethyl acrylate/sodium acryloyldimethyltaurate) copolymer, (ammonium acryloyldimethytaurate/VP) copolymer, (ammonium acryloyldimethyltaurate/carboxyethylammonium acrylate) crosspolymer, (acrylamide/sodium acryloyldimethyltaurate) copolymer, (dimethylacrylamide/sodium acryloyldimethyltaurate) crosspolymer (sodium acrylate/acryloyldimethyltaurine/dimethylacrylamide) crosspolymer, (sodium acryloyldimethyltaurate/VP) crosspolymer, ammonium polyacryloyldimethyltaurate, (sodium acrylate/sodium acryloyldimethyltaurate) copolymer and sodium polyacryloyldimethyltaurate, and mixtures thereof. In a particular and preferred composition, for further increased stability of the cosmetic composition, (hydroxyethyl acrylate/sodium acryloyldimethyltaurate) copolymer is used. Here, “VP” refers to vinylpyrrolidone.

The total content of component(s) (d) ranges preferably from 0.5 to 5 mass % and more preferably from 1 to 4 mass % based on the total mass of the composition. If the total content of component(s) (d) is within this range, the stability of the cosmetic composition will be even more excellent.

### Nonionic Surfactant (Optional)

In a particular embodiment, the cosmetic composition of the invention may also comprise a nonionic surfactant (e). If the cosmetic composition comprises component (e), the stability of the cosmetic composition will be even more excellent. Component (e) may be a single one used alone, or two or more may be used in combination.

Examples of nonionic surfactants according to the invention include glycerin fatty acid esters (for example, glyceryl myristate, glyceryl stearate and glyceryl oleate), sorbitan fatty acid esters (for example, sorbitan monostearate, sorbitan monooleate, sorbitan tristearate and sorbitan trioleate), sucrose fatty acid esters, polyoxyethylene alkyl ethers (for example, polyoxyethylene lauryl ether, polyoxyethylene cetyl ether, polyoxyethylene stearyl ether and polyoxyethylene oleyl ether), polyoxyethylene alkylphenyl ethers, alkyl glucosides (for example, decyl glucoside, lauryl glucoside and cocoglycoside), polyethylene glycol fatty acids (for example, polyethylene glycol monolaurate and polyethylene glycol monostearate) and polyoxyethylene sorbitan fatty acids (for example, polyoxyethylene sorbitan monostearate, polyoxyethylene sorbitan monooleate, polyoxyethylene sorbitan tristearate and polyoxyethylene sorbitan trioleate), and polyglycerin fatty acid esters (for example, polyglyceryl-10 myristate, polyglyceryl-10 stearate and polyglyceryl-10 oleate), and mixtures thereof. In a particular and preferred embodiment, component (e) is at least one selected from the group consisting of glycerin fatty acid esters and polyethylene glycol fatty acids, and more preferably a combination of glyceryl stearate and polyethylene glycol stearate, and mixtures thereof, for more excellent stability of the cosmetic composition.

The total content of component(s) (e) ranges preferably from 0.5 to 5 mass % and more preferably from 1 to 4 mass % based on the total mass of the composition. If the total content of component(s) (e) is within this range, the stability of the cosmetic composition will be even more excellent.

### Aqueous Medium

The cosmetic composition of the present invention contains an aqueous medium (f). Component (f) may consist of water alone, or it may also include a solvent that is soluble in water.

The water used in the present invention may be distilled water, purified water, hot spring water, deep water, or plant-derived steam distilled water such as lavender water, rose water or orange flower water.

Examples of solvents that are soluble in water according to the present invention include mono-alcohols such as methanol, ethanol, propyl alcohol, isopropyl alcohol and phenoxyethanol; and polyols such as ethylene glycol, 1,3-propanediol, propylene glycol, butylene glycol, pentylene glycol, hexylene glycol, dipropylene glycol, polyethylene glycol, glycerin, diglycerin, polyglycerin, sorbitol, xylitol and mannitol, and mixtures thereof. In a particular embodiment, the composition comprises water and polyols, preferably water and one or more polyols such as glycerin, 1,3-propanediol and mixtures thereof. The solvent that is soluble in water may be a single one used alone, or two or more may be used in combination.

The total content of component(s) (f) ranges preferably from 5 to 50 mass % and more preferably from 10 to 40 mass % based on the total mass of the composition.

The cosmetic composition of the invention may also contain, in addition to these components, oil agents, pH regulators, chelating agents, antioxidants, antiseptic agents, active to obtain an aqueous mixture. In a particular embodiment, the cosmetic composition of the invention does not contain any additional hydrocarbon oil which is solid or semi-solid at 30° C. including paraffin, petrolatum and the like. In particular, the cosmetic composition contains less than 5% by weight of hydrocarbon oil which is solid or semi-solid at 30° C. including paraffin, petrolatum and the like, in particular less than 1% by total weight of the composition, and preferably does not contain any hydrocarbon oil which is solid or semi-solid at 30° C. including paraffin, petrolatum and the like.

In a particular embodiment, the cosmetic composition can be prepared by the following steps, as an example.
1) Component(s) (c), component(s) (f) and other aqueous components as necessary are combined, heated at 70 to 80° C. (preferably at 75° C.) and stirred (preferably at a rotational speed of 100 to 300 rpm, in particular with Rayneri turbotest mixer from VMI), and then component(s) (d) is added and the mixture is heated at 70 to 80° C. (preferably at 75° C.) and stirred (preferably at a rotational speed of 100 to 300 rpm, in particular with Rayneri turbotest mixer from VMI) to obtain an aqueous mixture.
2) Component(s) (a), optional component(s) (e) and other oil components as necessary are mixed, heated at 70 to 80° C. (preferably at 75° C.) and stirred (preferably at a rotational speed of 100 to 300 rpm) to obtain an oil-based mixture.
3) The oil-based mixture obtained in 2) is added to the aqueous mixture obtained in 1), and the resulting mixture is heated at 70 to 80° C. (preferably at 75° C.) and stirred (preferably at a rotational speed of 300 to 600 rpm, in particular with Rayneri turbotest mixer from VMI).
4) Component(s) (b) and other components (such as an alkali metal hydroxide) as necessary are added to the mixture obtained in 3), and the resulting mixture is stirred (preferably at a rotational speed of 200 to 600 rpm, in particular with Rayneri turbotest mixer from VMI) and cooled to room temperature.

The cosmetic composition of the present invention can be suitably used as a cleanser since it is a fatty acid soap-type cosmetic composition.

The invention also relates to a cosmetic process for cleansing making-up keratinic materials, comprising the application onto keratinic materials, in particular onto skin, of the cosmetic composition as defined above. The cosmetic composition of the invention can be suitably applied to skin other than the scalp, and more preferably the face, body and hands or feet.

### Examples

The present invention will now be explained in specific detail through the following examples, with the understanding that the invention is not limited by the examples. Unless contrary, the percentage % are expressed in mass (weight) of compound per total mass (weight) of the composition.

### (1) Preparation of Cleanser

Cleansers were prepared by the following method, based on the compositions listed in Table 1.

Each of the components in component (c) and component (f) were mixed at 75° C., and then component (d) was added and the mixture was stirred at 75° C. to obtain an aqueous mixture. Each of the components in component (a) were then mixed in with component (e) at 75° C. to obtain an oil-based mixture. After adding the oil-based mixture to the aqueous mixture and cooling down to 55° C. potassium hydroxide was added, the mixture was stirred at 55° C., component (b) was further added and stirring was continued at 45° C. Each mixture was cooled to room temperature to obtain a cleanser.

### (2) Evaluation of Foaming Ability

An evaluation panel of 8 cosmetic experts evaluated each of the cleansers in terms of “speed of making foam” and “quantity of foam”. Each of the cleansers were graded according to the following A to C.
A: Over 6 experts were satisfied with foaming ability (‘good foaming ability’=good)
B: 4 to 5 experts were satisfied with foaming ability (‘medium foaming ability’=medium)
C: Less than 4 experts were satisfied with foaming ability (‘low foaming ability’=bad)

### (3) Evaluation of Non-Dryness

The same panel of 8 cosmetic experts also evaluated each of the cleansers in terms of “no feel dryness after rinsing off”. Each of the cleansers were graded according to the following A to C.
A: Over 6 experts could not feel dryness after rinsing off (‘no feel dryness’=good).
B: 4 to 5 experts could not feel dryness after rinsing off (‘low feel dryness’=medium).
C: Less than 4 experts could not feel dryness after rinsing off (‘feel dryness’=bad).

### (4) Stability Evaluation

Each cleanser was housed in a transparent container and the covering was sealed, after which it was stored at 45° C. for 1 month and an evaluation of “Y” (Yes=stable) was assigned when no separation between the oil phase and aqueous phase was observed, while an evaluation of “N” (No=unstable) was assigned when separation between the oil phase and aqueous phase was observed.

TABLE 1

| | | | | | | | | Comparative | Comparative | Comparative |
|---|---|---|---|---|---|---|---|---|---|---|
| | | | | | | | | Example 1 | Example 2 | Example 3 |
| | | | | | | | | Without | Without | Without |
| | | | | | | | | component | component | component |
| | Ingredient | Example 1 | Example 2 | Example 3 | Example 4 | Example 5 | Example 6 | b) | c) | d) |
| (a) | Lauric acid | 5.00 | 5.00 | 5.00 | 5.00 | 5.00 | 5.00 | 5.00 | 5.00 | 5.00 |
| | Myristic acid | 12.00 | 12.00 | 12.00 | 9.00 | 12.00 | 12.00 | 12.00 | 12.00 | 12.00 |
| | Stearic acid | 10.00 | 10.00 | 10.00 | 10.00 | 10.00 | 10.00 | 10.00 | 10.00 | 10.00 |
| (b) | Polyquaternium-7 | 1.00 | — | — | — | — | — | — | — | — |
| | Polyquaternium-22 | — | 1.00 | 1.00 | 1.00 | 1.50 | 1.00 | — | 1.50 | 1.00 |
| (c) | Sodium lauryl glycol | 3.00 | 3.00 | — | — | — | — | — | — | — |
| | carboxylate | | | | | | | | | |
| | Sodium lauroyl | — | — | 2.50 | — | — | — | — | — | — |
| | glutamate | | | | | | | | | |
| | Sodium methyl cocoyl | — | — | — | 3.00 | 3.90 | — | 3.00 | — | 3.90 |
| | taurate | | | | | | | | | |
| | Cocamidopropyl | — | — | — | — | — | 3.00 | — | — | — |
| | betaine | | | | | | | | | |
| (d) | Hydroxypropyl | 1.20 | 0.50 | 0.50 | 0.50 | — | 0.50 | 0.50 | — | — |
| | methylcellulose | | | | | | | | | |
| | stearoxy ether | | | | | | | | | |
| | Hydroxyethyl | — | — | — | — | 3.40 | — | — | 3.40 | — |
| | acrylate/sodium | | | | | | | | | |
| | acryloyldimethyl | | | | | | | | | |
| | taurate copolymer | | | | | | | | | |
| (e) | Glyceryl stearate and | 2.00 | 2.00 | 2.00 | 2.00 | 3.00 | 2.00 | 3.00 | 3.00 | 3.00 |
| | PEG-100 stearate | | | | | | | | | |
| (f) | Water | Balance | Balance | Balance | Balance | Balance | Balance | Balance | Balance | Balance |
| | Glycerin | 10.00 | 10.00 | 10.00 | 10.00 | 10.00 | 10.00 | 10.00 | 10.00 | 10.00 |
| | Propanediol | 3.00 | 3.00 | 3.00 | 3.00 | 3.00 | 3.00 | 3.00 | 3.00 | 3.00 |
| | Preservatives | q.s. | q.s. | q.s. | q.s. | q.s. | q.s. | q.s. | q.s. | q.s. |
| | PEG-8 | — | — | — | — | 5.00 | — | — | 5.00 | — |
| | Potassium hydroxide | 4.80 | 4.80 | 4.80 | 4.50 | 5.10 | 4.80 | 5.10 | 4.80 | 5.10 |
| Evaluation | Foaming ability | A | A | A | A | A | A | A | B | A |
| results | Non-dryness | A | A | A | A | A | A | C | C | A |
| | Stability | Y | Y | Y | Y | Y | Y | N | Y | N |

The results presented in the Table 1 show that the combination of (a) one or more higher fatty acids of 12 to 24 carbon atoms or their alkali metal salts, (b) at least one cationic polymer selected from the group consisting of polyquaternium-7 and polyquaternium-22, (c) an anionic surfactant and/or an amphoteric surfactant, and (d) at least one thickener selected from the group consisting of hydrophobized hydroxypropyl methylcellulose, and (meth)acrylic polymers comprising a (meth)acryloylalkyltaurine or its salt as a structural unit, provide a cleansing composition having excellent foaming ability, with stability and ‘non-dryness’ feel after application, in comparison with other comparative examples.

1. A cosmetic composition comprising:
(a) one or more higher fatty acids of 12 to 24 carbon atoms or their alkali metal salts,
(b) at least one cationic polymer selected from the group consisting of polyquaternium-7 and polyquaternium-22,
(c) an anionic surfactant and/or an amphoteric surfactant, and
(d) at least one thickener selected from the group consisting of hydrophobized hydroxypropyl methylcellulose, and (meth)acrylic polymers comprising a (meth)acryloylalkyltaurine or its salt as a structural unit.

2. The cosmetic composition according to claim 1, wherein the one or more higher fatty acids of 12 to 24 carbon atoms or their alkali metal salts are two or more selected from the group consisting of lauric acid, myristic acid, palmitic acid, stearic acid and their alkali metal salts.

3. The cosmetic composition according to claim 1, wherein the total content of component(s) (a) ranges from 15 to 50 mass % based on the total mass of the composition.

4. The cosmetic composition according to claim 1, wherein the cationic polymer is polyquaternium-22.

5. The cosmetic composition according to claim 1, wherein the total content of component(s) (b) ranges from 0.2 to 3.5 mass % based on the total mass of the composition.

6. The cosmetic composition according to claim 1, wherein the anionic surfactant and/or amphoteric surfactant is one or more selected from the group consisting of alkyl ether carboxylic acid salts, N-acylglutamic acid salts, N-methyl-N-acyltaurine salts, and betaine aminoacetate-type amphoteric surfactants.

7. The cosmetic composition according to claim 1, wherein the anionic surfactant and/or amphoteric surfactant is one or more selected from the group consisting of sodium laurylglycol carboxylate, sodium lauroyl glutamate, sodium cocoyl methyltaurate, and cocamidopropyl betaine.

8. The cosmetic composition according to claim 1, wherein the total content of component(s) (c) ranges from 5 to 20 mass % based on the total mass of the composition.

9. The cosmetic composition according to claim 1, wherein the hydrophobized hydroxypropyl methylcellulose is hydroxypropyl methylcellulose stearoxy ether, and the (meth)acrylic-based polymer is (hydroxyethyl acrylate/sodium acryloyl dimethyltaurate) copolymer.

10. The cosmetic composition according to claim 1, wherein the total content of component(s) (d) ranges from 0.5 to 5 mass % based on the total mass of the composition.

11. The cosmetic composition according to claim 1, which further comprises (e) a nonionic surfactant.

12. The cosmetic composition according to claim 11, wherein the nonionic surfactant is at least one selected from the group consisting of glycerin fatty acid esters and fatty acid polyethylene glycols, and mixtures thereof.

13. The cosmetic composition according to claim 1, which further comprises (f) an aqueous medium.

14. The cosmetic composition according to claim 1, which is a cleanser.

15. A cosmetic process for cleansing keratinic materials, comprising the application onto keratinic materials, the cosmetic composition as defined in claim 1.

16. The cosmetic composition according to claim 13, wherein the aqueous medium comprises water and one or more polyols.
